(19) United States
(12) Patent Application Publication
(10) Pub. No.: US 2003/0147843 A1
(43) Pub. Date: Aug. 7, 2003

(54) Support material loaded with volatile substances, method for producing the same and use thereof

(76) Inventors: Dietmar Lange, Vallendar (DE); Peter Schlimm, Montabaur (DE)

Correspondence Address: FULBRIGHT & JAWORSKI, LLP, 1301 MCKINNEY, SUITE 5100, HOUSTON, TX, 77010-3095, US

(21) Appl. No.: 10/220,461

(22) PCT Filed: Feb. 28, 2001
(86) PCT No.: PCT/DE01/00758

(30) Foreign Application Priority Data
Mar. 2, 2000 (DE) 100 10 210.7

(51) Int. Cl. A61L 9/04; C04B 33/24

(52) U.S. Cl. 424/76.4; 501/143

(57) ABSTRACT

Support material from porous ceramic granulate, produced from a mixture of 45 to 80 parts by weight clay, 0 to 50 parts by weight calcite, quartz sand, dolomite, feldspar, chamotte and/or sepiolite, 15 to 30 parts by weight water, 0.01 to 0.03 parts by weight deflocculant, 0.001 to 0.01 parts by weight surfact and optionally agents counteracting the deflocculant and foamed under an overpressure of at least 0.3 bar prior to baking and filled or charged with at least one volatile substance.

The invention relates to porous support materials charged or filled with volatile substances and which are able to release the absorbed, volatile substances into the ambient air over a long period of time. The invention also relates to methods for the manufacture of porous support materials charged or filled with volatile substances.

For the release of volatile substances into the ambient air more particularly plastic foams are known, which are impregnated with perfumes.

Compared with this prior art, the problem of the present invention is to provide an environmentally friendly support having absorbed, volatile substances and which can release the volatile substances contained at an approximately constant rate over a long time period, as well as to a method for the manufacture thereof

The solution according to the invention comprises liquid, volatile substances being applied to porous ceramic material, which has a suitable pore size or pore structure for receiving said substances. The volatile substances can also be applied in aqueous and/or organic solution. Suitable volatile substances are aromatic substances, insect repellents and volatile substances able to react with disturbing odorous substances.

The support matrix according to the invention is porous ceramic material, which has a suitable pore size and/or characteristics, such as the ceramic granulate produced according to EP 427 704 B 1. This support material is manufactured from 45 to 80 parts by weight clay, 0 to 50 parts by weight calcite, quartz sand, dolomite, feldspar, chamotte and/or sepiolite, 15 to 30 parts by weight water, 0.01 to 0.03 parts by weight deflocculant and 0.001 to 0.01 parts by weight surfactant, which are mixed and foamed in a dispersing unit at a pressure of 1 to 10 bar and a temperature ranging from ambient temperature to 95° C. Optionally for the manufacture of a high-viscosity, thixotropic and naturally stable foam suspension it is possible to use agents counteracting the deflocculant and then the foamed mixture is shaped into clay articles. The shaped clay articles are baked between 600 and 1500° C., preferably between 700 and 800° C. The uniform pore structure with pore sizes of <1 mm is obtained by foaming the starting mixture at an overpressure of at least 0.3 bar, the pressure being maintained up to discharge from the nozzle.

The advantages of this porous ceramic support with respect to environmental compatibility are that it can weather and can even be composted. On use the support filled with the volatile substances is dimensionally stable and the support is not flammable. The very large internal surface or the large internal volume of the porous ceramic support makes it possible to absorb a large quantity of volatile substances, which can be released via the uniform pore structure over a long time period. The filled support material is consequently able to release the absorbed, volatile substances at a uniform rate and over a long time period into the ambient air.

It has surprisingly been found that the porous ceramic support material is also suitable for absorbing lipophilic, volatile substances, although the ceramic material itself has no organic polymer surface with lipophilic properties and instead, due to its composition, is highly polar. Without being bound by it as a limitation, it is assumed that the favourable pore structure of the ceramic material permits the absorption of very large quantity of volatile substances and their uniform, long-lasting release into the ambient air.

The application of or impregnation with volatile substances can take place by spraying the volatile substances or their solutions onto the porous support material. As a function of the volatile substances used, suitable solvents are water and/or organic solvents. After spraying the volatile substances or their solutions onto the ceramic support, the solvent can be removed by evaporation at ambient temperature or elevated temperature. Such a spraying of the ceramic support can be repeated in order to obtain a higher charging or filling of the support material.

A particularly favourable method for the application of volatile substances or their solutions to the support material comprises spraying the latter at reduced pressure, e.g. 200 mbar, preferably 40 mbar, applying a certain holding time of e.g. 7, preferably 5 and in particularly preferred manner 2 minutes at the reduced pressure and subsequently aerating the sprayed support material to ambient pressure. The pressure can be reduced before, during or after the application of the volatile substances or their solution to the support material. Solvents used for the application of the volatile substances can advantageously be evaporated under reduced pressure in order to solely retain in the support material the desired substances. Also when using a reduced pressure for the application of volatile substances to the support material, following the aerating to ambient pressure, any solvent used can be evaporated and optionally heating to above ambient temperature can take place. The application of volatile substances using reduced pressure can also be used several times in succession in order to obtain a higher filling of the ceramic support with the volatile substances. The multiple treatment of the porous support is particularly advantageous if the volatile substances are applied in solution.

The support materials filled with volatile support substances according to the invention can be used both as air fresheners and for improving, preferably avoiding unpleasant odours given off by waste or refuse collection points, such as e.g. dustbins or containers for collecting biological waste and packs suitable for reprocessing.

The present invention is further illustrated by the following examples:

### EXAMPLE 1

Perfume Composition for Use as an Air Freshener and Applied to Porous Support Material:

| | | |
|---|---|---|
| Electrolytic composition of natural and nature- | approx. 20 | wt. % |
| identically smelling oils, whereof 35 to 65% | | |
| natural oils and 35 to 65% nature-identical oils | | |
| Natural raw material-based surfactants or natural | approx. 10 | wt. % |
| surfactants | | |
| Water | to 100 | wt. % |

The composition of the odorous oils used can be adjusted as a function of the desired perfume. Particularly suitable are naturally occurring or nature-identical perfumes, such as in particular flower perfumes, freshly smelling aldehydes, fruity smelling esters, etc., as known to the perfume expert.

### EXAMPLE 2

Porous Support Material as the Support Matrix:

Ceramic granulate produced according to EP 427 704 from 45 to 80 parts by weight clay, up to 50 parts by weight calcite, quartz sand, dolomite, feldspar, chamotte, sepiolite as aggregate, 15 to 30 parts by weight water, 0.01 to 0.03 parts by weight deflocculant and 0.001 to 0.1 parts by weight surfactant, which are mixed and foamed in a dispersing unit at a pressure of 1 to 10 bar and a temperature ranging from ambient temperature to 95° C. and baked as shaped clay articles at between 700 to 800° C. (as described herein-before) is sprayed with a perfume composition at 200 mbar, kept at this reduced pressure for 5 minutes and ten aerated to ambient pressure.

### EXAMPLE 3

Use of Porous Support Material Filled with a Perfume Composition According to Example 1:

A perfume composition according to example 1 is introduced in a proportion of 10 to 30 wt. %, preferably 20 to 30 wt. % and in particularly preferred manner 22 to 25 wt. % into the support matrix. In this use form, surprisingly the odorous constituents are completely absorbed by plant, if its roots are at least partly in contact with the filled support, but without the roots being damaged.

### EXAMPLE 4

Use of Porous Support Materials Filled with a Perfume Composition for Avoiding Unpleasant Smells in Collecting Barrels for Biological Waste (so-called bio-barrels)

In the case of 70 collecting barrels with a 200 litre volume for biological waste, in each case approximately 40 ml of the porous support material according to the invention and filled in accordance with example 2 with a perfume composition of approximately 8 to 10 wt. %, were fixed to the inside of the lid. To permit the passage or evaporation of the perfume constituents, the filled support material was placed in a wire basket fixed to the inside of the barrel lid. For housing the porous support material charged with perfume composition in accordance with the invention are also suitable screen bags or bags made from permeable fabric and porous plastics.

According to the evaluation of the user, the odour release in the case of 89% of the biological waste collecting barrels was after 2 weeks adequately reduced to remain below a disturbing odour emission.

The features of the invention disclosed in the description, drawings and claims can be essential to the implementation of the different embodiments of the invention, both singly and in random combination.

1. Support material of porous ceramic granulate produced from a mixture of 45 to 80 parts by weight clay, 0 to 50 parts by weight calcite, quartz sand, dolomite, feldspar, chamotte and/or spiolite, 15 to 30 parts by weight water, 0.01 to 0.03 parts by weight deflocculant, 0.001 to 0.1 parts by weight surfactant and optionally agents counteracting the deflocculant and which was foamed under an overpressure of at lest 0.3 bar prior to baking and filled with at least one volatile substance.

2. Method for the manufacture of support material according to claim 1, characterized in that the volatile substances are applied at ambient pressure.

3. Method for the manufacture of support material according to claim 1, characterized in that the volatile substances are applied at a reduced pressure of 200 mbar.

4. Method for the manufacture of support material according to claim 1, characterized in that the volatile substances are applied at a reduced pressure of 40 mbar.

5. Method for the manufacture of support material according to one of the claims 2 to 4, characterized in that the volatile substances are applied in solution.

6. Method for the manufacture of support material according to one of the preceding claims, characterized in that the volatile substances are applied to the support material in a composition containing 10 to 90 wt. % of volatile substance, up to 15% surfactant and up to 70% water.

7. Use of support material manufactured according to the method of the preceding claims as an air freshener.

8. Use of support material manufactured according to a method according to the preceding claims for improving the odour emitted by refuse or biological waste collecting containers.

9. Use of the support material manufacture according to a method of the preceding claims as an additive to compost.

10. Use according to claim 9, in which the perfume composition contains at least one surfactant.
